(19) United States
(12) Patent Application Publication
(10) Pub. No.: US 2020/0069482 A1
(43) Pub. Date: Mar. 5, 2020

(54) pH Indicator Dressing for Monitoring of Wound and Infection

(71) Applicant: UNIVERSITY OF SOUTH CAROLINA, COLUMBIA, SC (US)

(72) Inventors: EHSAN JABBARZADEH, COLUMBIA, SC (US); SARA ESLAMBOLCHIMOGHADAM, COLUMBIA, SC (US)

(21) Appl. No.: 16/560,125

(22) Filed: Sep. 4, 2019

Related U.S. Application Data
(60) Provisional application No. 62/727,214, filed on Sep. 5, 2018.

(51) Int. Cl. A61F 13/42; A61B 5/00; A61F 13/00; B01J 20/26; B01J 20/28; B01J 20/32

(52) U.S. Cl. CPC A61F 13/42; A61B 5/445; A61F 13/00017; A61F 13/00063; A61F 13/00038; A61F 13/00042; B01J 20/28047; B01J 20/3244; B01J 20/3272; A61F 2013/427; A61F 13/00012; B01J 20/267

(57) ABSTRACT

Wound dressings, methods for forming the wound dressings, and methods for using the wound dressings are described. Wound dressings include a polymeric composition that includes a biocompatible polymer and a pH indicating agent. The polymeric composition can be at a wound contacting surface of a wound dressing in the form of, e.g., a fiber, a hydrogel, or a microsphere. The color of the polymeric composition as determined by the pH indicating agent can indicate the presence of infection or a chronic wound state. The wound dressings can be used for early detection of healing abnormalities in acute, chronic, or burn wounds.

### CROSS REFERENCE TO RELATED APPLICATION

This application claims filing benefit of U.S. Provisional Patent Application Ser. No. 62/727,214, having a filing date of Sep. 5, 2018, entitled “pH Indicating Dressing for Wound Biomonitoring,” which is incorporated herein by reference for all purposes.

### FEDERAL RESEARCH STATEMENT

This invention was made with Government support under Grant No. 1811949, awarded by the National Science Foundation, and under Grant No. R03 EB026813, awarded by the National Institutes of Health. The Government has certain rights in the invention.

### BACKGROUND

Proper wound care varies with the type of wound. For instance, acute wounds, i.e., wounds due to trauma, surgery, etc., will normally progress through the physiological stages of inflammation, tissue formation, and remodeling if the wound is kept clean. In contrast, chronic wounds, such as leg ulcers, foot ulcers, and pressure sores or decubitus ulcers caused by sustained external skin pressure, do not heal normally and require intensive levels of care. Burn wounds can likewise require intensive long-term treatment. Even with the best of care, any wound, even an apparently inconsequential acute wound, can become infected with potentially deadly results if the infection is not properly identified and treated.

In clinical practice, diagnosis of wound infection is often based on initial determination of secondary parameters, such as odor, presence of local pain, heat, swelling, discharge, and redness. For instance, a wound may be assessed visually, length and depth measurements may be taken, and digital photography may be used to track the visual condition and size of a wound. Unfortunately, many of these clinical indicators have a low predictive value of infection, and confirmation of the suspected infection is required before treatment can begin as misdiagnosis of wound infection risks inappropriate antibiotic prescribing, antibiotic resistance, and unnecessary treatment side effects.

Swabbing of fluid from the area of the suspected infection followed by microbiology testing is a standard option for confirmation of bacterial colonization and identification of the strains associated with infection. Unfortunately, this process is time consuming and the time lag from onset of infection to detection is one of the biggest drawbacks associated with existing clinical diagnostics, as delay in diagnosis can delay treatment and negatively affect treatment outcome. Moreover, this process is labor-intensive and the multiple steps required in traditional swab-based diagnoses increase the potential for error, such as sampling errors, delays in transport of the swabs, errors in analytical procedures, and/or errors in reporting. While wound swabs have proven useful, the wait for testing results of the sample and the potential for introduction of error has limited their usefulness in the clinical setting, particularly for outpatients.

The pH of biological fluids can provide a good determination of the status of a wound. The normal pH of intact skin ranges from about 4.8 to about 6.0. When a wound occurs, the skin acidic milieu and pH is disrupted, exposing the more neutral pH of the underlying tissue (generally about pH 7.4). Over the course of successful healing and re-epithelialization, the initial, more neutral, pH of the wound will drop due to various factors including hypoxia and increased production of lactic acid. An acidic pH environment is considered to be beneficial during healing by increasing fibroblast proliferation and migration, promoting epithelization, regulating bacterial colonization, and facilitating the release of oxygen.

If wound healing is delayed, the pH can oscillate and become increasingly alkaline over time. The pH of a wound can affect many factors of the local environment including oxygen release, angiogenesis, protease activity, macrophage and fibroblast activity, and bacterial toxicity. An alkaline wound environment can be indicative of a chronic wound (chronic wound environments have been reported in the range of pH 7.15 to 8.93), and wounds of any sort having an elevated alkaline pH have been shown to have lower rates of healing than wounds in which the pH is lower. Alkaline wound environments are also more likely to become infected and many wounds, such as chronic leg ulcers, are often colonized by intestinal, oral and resident dermal microorganisms. High pH can thus be an indicator of an existing infection, as pH increase from normal baseline is more favorable for bioburden of pathogenic microorganisms. Moreover, some bacteria produce ammonia, which in itself is necrotizing, and which can impair oxygenation of the tissues by further raising the pH and creating a self-sustaining cycle.

It is known that the measurement of pH facilitates early detection of infection, which can enable early therapeutic intervention and improve outcomes. Researchers have developed multiple pH indicator compositions and devices. For instance, U.S. Pat. Nos. 5,217,444; 5,660,790; 5,823,953; 5,897,834; 5,910,447; and 6,106,461 and U.S. Pat. App. Pub. Nos. 2009/0275071; 2015/0308994; and 2016/0106880 disclose devices and methods for analyzing the pH of various bodily secretions. Commercial examples of pH sensitive products include SwabCheck™ (Sigma), which includes a cellulose swab designed to collect a sample that is then transferred to an incubation tube; Amnicator™ which includes disposable swabs impregnated with nitrazine yellow dye; and VS-SENSE PRO™, which is a qualitative, visually readable swab for the evaluation vaginal fluid.

Unfortunately, many existing colorimetric pH indicator systems include the pH indicator simply adsorbed to a surface of the indicator as such are subject to dye leaching. In addition, many systems require a reagent to extract the biological materials from the collection device and to develop a detectable color. Such issues add to cost, complication, and potential error. Conventional pH indicators are also difficult to utilize as they require regular calibration, include fragile electrodes that must remain wet, and lack flexibility often necessary to access tissue or fluids of interest.

Despite the wide variety of wound dressings available, there is an unmet need in the wound care sector for wound dressings that can incorporate diagnostic reagents that enable early diagnosis of wound complications such as chronic wound or infection, and which permit diagnosis prior to manifestation of clinical symptoms of complications.

### SUMMARY

According to one embodiment, disclosed is a wound dressing that includes a wound contacting surface and an absorbent substrate at the wound contacting surface. The absorbent substrate includes a polymeric composition that includes a biocompatible polymer and a pH indicator. For instance, the absorbent substrate can include a hydrogel, a fiber, or a particle that can include the polymeric composition. The pH indicator can exhibit a detectable color in the visible spectrum that is indicative of the pH of a biological fluid (e.g., a wound exudate) in contact with the absorbent substrate.

Also disclosed is a method for forming a wound dressing. A method can include forming a polymeric precursor that includes a pH indicator and a biocompatible polymer. The method can also include solidifying the precursor to form a polymeric composition that incorporates the biocompatible polymer and the pH indicator. The method can also include forming an absorbent substrate that can be a wound contacting surface of a wound dressing, with the absorbent substrate including the polymeric composition.

A method for treating a wound is also disclosed. For instance, a treatment method can include applying a wound dressing as described herein to a wound and visually examining the wound contacting surface of the wound dressing to determine a color of the pH indicator. The method can be utilized in wound treatment to facilitate early diagnosis and treatment of chronic wounds or infected wounds.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1 presents a schematic illustration of exemplary formation processes as may be used in forming a wound dressing as described herein.

FIG. 2 illustrates the colorimetric transition of a bromothymol blue-containing fibrous substrate in response to different pH. The pH sensitive substrate turned light green, dark green, and blue in 10 seconds when exposed to different pH buffer.

FIG. 3 illustrates the colorimetric transition of a litmus-containing substrate in response to buffer pH 9.2. The purple pH sensitive beads within the substrate turned blue when exposed to the basic pH.

FIG. 4 illustrates the colorimetric transition of a bromothymol blue-containing fibrous substrate in response to different concentrations of E. coli after 10 seconds of contact.

FIG. 5 illustrates the colorimetric transition of an anthocyanin-containing substrate in response to P. aeruginosa. The pH sensitive beads turned green after 1 hour of contact.

FIG. 6 illustrates the colorimetric transition of litmus-containing substrate in response to P. aeruginosa. The pH sensitive beads turned blue after 1 hour of contact.

FIG. 7 illustrates the colorimetric transition of bromothymol blue-containing fibrous substrate in response to mature bacterial biofilm grown on porcine tissue.

FIG. 8 graphically illustrates the results of a cytotoxicity analysis of a fibrous polyurethane-based substrate incorporating a pH indicator.

FIG. 9 presents an image of control and test substrates used in the cytotoxicity analysis of FIG. 8.

FIG. 10 graphically illustrates the results of a cytotoxicity analysis of fibrous polyurethane-based substrates incorporating various concentrations of a pH indicator in the substrate after 24 hours of exposure.

FIG. 11 graphically illustrates extraction results using Dulbecco's Modified Eagle Medium (DMEM) as extractant for 24 h. As shown, pH indicator release from the substrate was in the safety range as indicated in FIG. 10.

Repeat use of reference characters in the present specification and drawings is intended to represent the same or analogous features or elements of the present invention.

### DETAILED DESCRIPTION

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment.

The disclosure is generally directed to wound dressings that can be utilized for fast and reliable detection of potential healing issues in a wound. More specifically, disclosed are wound dressings, methods for forming the wound dressings, and methods for utilizing the wound dressings that incorporate pH indicators within the dressings. The wound dressings can provide a visually readable wound-contacting surface that is accessible to clinicians, patients, or others who wish to quickly and reliably evaluate wound status. The wound dressings include a pH indicator as a component of a polymeric composition used in forming an absorbent substrate of the wound dressing. Beneficially, by retaining a pH indicator within a polymeric composition of the wound dressings, the pH indicator can be prevented from leaching from the dressing and/or dispersing in the wound area, while still providing a biocompatible and pH indicating interface with the wound site. Retention of the pH indicator as a component of a polymeric composition can also prevent color change of the indicator due to unintended contact with a fluid other than the biological fluid of interest.

Considering the importance of wound pH, continually monitoring the pH of wound exudate as is possible with disclosed wound dressings can be extremely helpful in guiding wound management practices and in determining effective treatment strategies. Disclosed dressings can be effective in treatment of any form of wound or skin abnormality including dermatology and cosmetic applications, as well as more traditional wound applications (acute wounds due to trauma, chronic wounds, burn wounds, etc.). The wound dressings can overcome disadvantages of previously known wound dressings by, e.g., providing a non-toxic composition that allows for monitoring of wound status through determination of wound pH and, in some embodiments, by providing direct confirmation of bacterial infection.

The wound dressings can provide a one-step test with results that can be interpreted by simply looking at the dressing color. The test is very simple and convenient, and visual results appear within seconds or minutes of contact, without need for any inspection equipment. Moreover, in some embodiments, the disclosed wound dressings can exhibit absorbency without wound desiccation so as to maintain desirable fluid balance at the wound site. The wound dressings can also be easy to apply and remove without fear of damage to either the wound area or the wound dressing, and can provide hemostasis and protection while maintaining gas permeability so as to promote wound healing. Moreover, the wound dressings can exhibit mechanical characteristics that accommodate movement while in use without disintegration of the wound dressings and do not restrict patient motion.

The pH indicator of a wound dressing can be any suitable colorimetric pH indicator that is capable of exhibiting a relatively rapid color change in the visible spectrum (light having a wavelength of from about 380 nm to about 740 nm) with change in pH. A pH indicator can in one embodiment detect pH between about pH 0 and about pH 14, though a pH indicator that detects pH within a smaller range is also encompassed. For instance, a pH indicator may be utilized that detects pH between about pH 5 and about pH 12 or about pH 6 and about pH 10 (e.g., a pH range expected in wound care). A suitable pH indicator can indicate changes in pH by way of a color change along the visual spectrum, with visually identifiable colors in the spectrum being associated with a particular pH. In addition, a pH indicator can exhibit a color change relatively rapidly, generally within a few minutes, e.g., about 5 minutes or less or about 1 minute or less in some embodiments.

Examples of suitable pH indicators can include, without limitation, litmus, anthocyanin, nitrazine yellow, brilliant yellow, cresol red, bromocresol purple, chlorophenol red, bromothymol blue, thymol blue, bromoxylenol blue, neutral red, phenol red, xylenol blue, m-cresol purple, orcein, erythrolitmin (or erythrolein), azolitmin, spaniolitmin, leucoorcein, and leucazolitmin, as well as combinations of indicators.

The wound dressings include the pH indicator as a component of a polymeric composition that can be utilized in forming at least a portion of a wound dressing. The polymer(s) of the polymeric composition are not particularly limited and can encompass any biocompatible polymer. The preferred polymers for an embodiment can generally depend upon the desired form of the polymeric composition in the wound dressing. For instance, in those embodiments in which the polymeric composition is intended to be utilized in a fibrous form, e.g., in a woven or non-woven pad, gauze, wadding, sheet, or the like, biocompatible polymers known for use in fiber forming technologies (fiber melt or solution spinning, fiber extrusion, electrospinning, etc.) can be preferred. Preferred biocompatible polymers for use in other applications, e.g., in forming a polymeric hydrogel, polymeric beads, etc., for inclusion in a wound dressing can vary as is known in the art, with preferred biocompatible polymers for particular applications being well within the understanding of one of ordinary skill in the art.

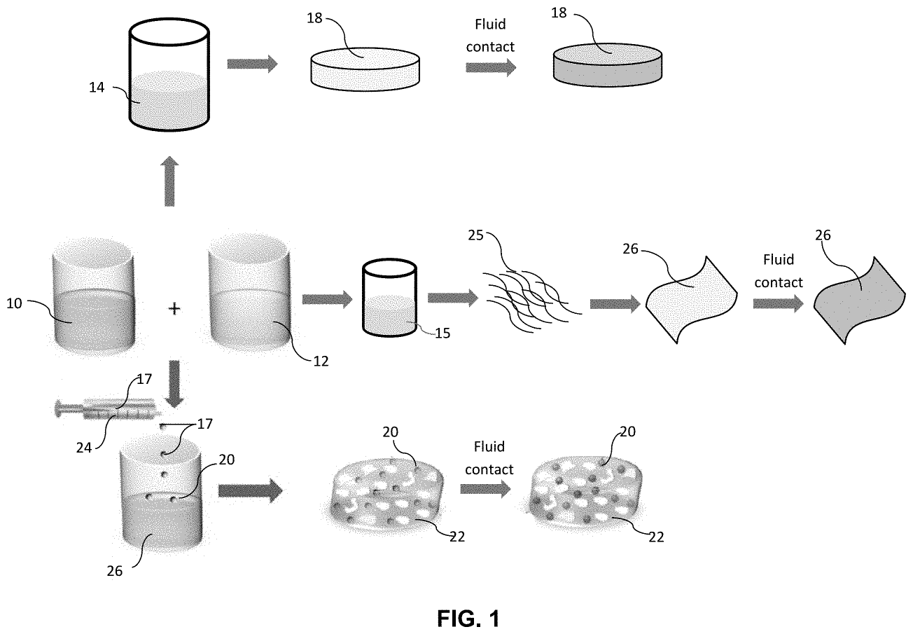

FIG. 1 illustrates several different embodiments for forming a wound dressing as described herein. Independent of the final form of the polymeric composition (e.g., fiber, hydrogel, etc.), a formation approach can include combination of a biocompatible polymer with a pH indicator to form a polymeric precursor. For instance, as illustrated in FIG. 1, a pH indicator 10 can be combined with a biocompatible polymer 12 to form a polymeric precursor such as a hydrogel precursor 14, a fiber precursor 15, or a microsphere precursor 17. Depending upon the nature of the pH indicator and the precursor, it may be preferred in some embodiments that the pH indicator is first dissolved by use of a suitable solvent, e.g., an aqueous or non-aqueous solvent (e.g., an alcohol such as ethanol or the like) and then combined with water to form a solution of the pH indicator 10. A solution including the pH indicator 10 can likewise include co-solvents or the like as are generally known in the art to provide for dissolution of the pH indicator 10.

Depending upon the nature of the various components and the final form of the polymeric composition, the pH indicator 10 and the polymer 12 can be combined with each in the form of a solution or in any other suitable form. For instance, in some embodiments, a polymer of choice can be dissolved or dispersed in a solution of a pH indicator to form a polymeric precursor solution. Alternatively, a pH indicator can simply be added to a polymer to form a polymeric precursor composition that can be in the form of a solution, a solid mixture, a melt, a suspension, or any other suitable composition type. Following combination, a polymeric precursor that includes both a pH indicator 10 and a biocompatible polymer 12 can be processed to form a polymeric composition that includes both the biocompatible polymer and the pH indicator.

In one embodiment, a polymeric precursor can be a hydrogel precursor solution 14 that can be crosslinked and shaped as desired to form a crosslinked hydrogel matrix 18 that incorporates the pH indicator. A hydrogel matrix 18 can be absorbent, and can maintain moisture at a wound while also absorbing excess fluids, as well as other undesirable materials exuded from the wound, such as dead leucocytes, epidermal cells, and dermal cells. In addition; a crosslinked hydrogel matrix 18 at a surface of a wound dressing can prevent or eliminate the formation of dry crusty scar tissue and can soften necrotic tissue at the wound site.

A hydrogel matrix 18 can be formed from one or more non-toxic, biocompatible hydrogel-forming polymers that include or can be modified to include crosslinkable functionality. The matrix polymer can include synthetic and/or natural hydrogel-forming polymers. By way of example, and without limitation, hydrogel matrix polymers can include alginates, collagen or derivatives thereof, cellulose or derivatives thereof, poly(lactic-co-glycolic acid) (PLGA) or derivatives thereof, polycaprolactone (PCL) or derivatives thereof, as well as combinations of different polymers, e.g., blends or copolymers thereof.

According to one embodiment, the biocompatible polymer of a hydrogel matrix 18 can include an alginate. Alginate is a naturally occurring anionic biocompatible polymer with low toxicity composed of a variety of alginic acids extracted from certain species of seaweeds. Alginate has been extensively investigated for many biomedical applications, including highly absorbent wound dressings. Alginate contains blocks of (1,4)-linked β-D-mannuronate (M) and α-L-guluronate (G) residues. Alginate has strong hydrophilicity, and as such, can form a highly absorbent wound dressing. Alginates are commercially available as pads, ropes, or ribbons from a variety of different suppliers. Alginate can be attractive for incorporation in a hydrogel matrix in one embodiment as it is known as an approved material for epidermal applications and has excellent biocompatibility and mechanical properties. Additionally, calcium alginate is known as a natural hemostat that can be removed from a wound site limited or no trauma and discomfort.

The hydrogel matrix polymer is not limited to alginates, however, and other non-toxic, biocompatible hydrogel-forming polymers can be utilized in conjunction with or alternative to an alginate. For instance, in one embodiment, a hydrogel matrix 18 can incorporate collagen, or a derivative thereof, for instance in the form of gelatin. Collagen is one of the main protein components of bone, cartilage, tendons, ligaments, and skin. Gelatin can be obtained from collagen by acidic or basic hydrolysis or thermal degradation of collagen, which leads to rupture of the collagen triple helix into the random coil structure of gelatin.

In one embodiment, the hydrogel matrix 18 can include a cellulose polymer or a derivative of cellulose (e.g., cellulose acetate, sodium carboxymethyl cellulose, ethylcellulose, nitrocellulose, bacterial cellulose, etc.). Cellulose is the most abundant polysaccharide, and it is inexpensive with good processability, renewability, and ease of physical and chemical modification. It has good mechanical properties, good hydrolytic stability, low toxicity, and excellent biocompatibility.

PCL, as may be incorporated in a hydrogel matrix 18, is a hydrophobic, semi-crystalline, resorbable, aliphatic polyester. The crystallinity decreases with increasing molecular weight, and PCL exhibits good solubility and low melting point (59° C.-64° C.), as well as excellent blend-compatibility, making it attractive for application in disclosed wound dressings. PCL can be biodegradable, but the degradation and resorption kinetics of PCL are relatively slow due to its hydrophobicity and high crystallinity, and as such, it can be beneficial in some embodiments as a component of disclosed hydrogels. PCL can be blended or co-polymerized with other polymers, such as PLA or PLGA, in order to modify its physical properties in a desirable fashion.

PLGA is a highly studied biodegradable polymer as may be incorporated in hydrogel matrix 18. In vivo, it is hydrolyzed into the non-toxic lactic acid and glycolic acid monomers. PLGA is commercially available in different molecular weights and copolymer compositions. The rate of biodegradation of a PLGA polymer can be controlled through selection of the copolymer ratio and molecular weight.

An hydrogel precursor solution 14 can generally include from about 0.5% w/v to about 20% w/v of the biocompatible polymer(s) of choice and from about 0.1% w/v to about 2% w/v of the pH indicator. In some embodiments, a hydrogel precursor solution 14 can include a crosslinking agent and/or a crosslink initiator. In some embodiments, one or both of a crosslinking agent and a crosslink initiator can be added to the hydrogel precursor solution 14 at the time of crosslinking.

Conventional biocompatible cross-linking agents—as are suitably used to provide the necessary mechanical stability and to control the properties of a hydrogel matrix 18—can be included in a hydrogel precursor solution (or combined with a precursor solution at the time of crosslinking). When included in the hydrogel precursor solution 14, the amount of crosslinking agent and/or crosslink initiator to be included will be readily apparent to those skilled in the art. For instance, a crosslinking agent can be included in an amount of from about 0.01% w/v to about 0.5% w/v, from about 0.05% w/v to about 0.4% w/v, or from about 0.08% to about 0.3% w/v, of the hydrogel precursor solution. Typical crosslinking agents can include, without limitation, tripropylene glycol diacrylate, ethylene glycol dimethacrylate, triacrylate, and methylene bis acrylamide. In one embodiment, a cationic crosslinking agent can be utilized. For example, a polyvalent elemental cation, such as Ca²⁺, Mg²⁺, Al³⁺, La³⁺, or Mn²⁺, can crosslink polymers of a hydrogel matrix.

A hydrogel precursor solution 14 may include a crosslink initiator. When included, a precursor solution 14 can include a crosslink initiator in standard amounts, e.g., up to about 5% w/v, for instance from about 0.002% w/v to about 2% w/v. In one embodiment, a hydrogel precursor solution 14 can include a photoinitiator, such as, and without limitation to, benzoyl radicals such as type I-α-hydroxy-ketones and benzilidimethyl-ketals (e.g., Irgacure 651, Irgacure 184 and Daracur 1173, as marketed by Ciba Chemicals), as well as combinations thereof.

In some embodiments, crosslinking can be initiated upon contact of a crosslinking agent with the hydrogel precursor solution 14 in conjunction with suitable crosslink initiation parameters as necessary (e.g., temperature or light). For instance, a hydrogel precursor solution 14 containing sodium alginate as the hydrogel polymer can be combined with a divalent cation (via e.g., a CaCl₂) solution) and crosslinking can spontaneously occur via ion exchange. In other embodiments, the hydrogel precursor solution 14 can be processed prior to crosslinking. For instance, a hydrogel precursor solution can be shaped via molding and water can be removed from the precursor solution 14 prior to crosslinking and production of the crosslinked hydrogel matrix 18.

As indicated in FIG. 1, due to the incorporation of a pH indicator in the hydrogel matrix 18, contact of the absorptive hydrogel matrix 18 with a biological fluid of interest, e.g., a wound extrudate, can lead to absorption of the fluid and contact of the fluid with the pH indicator retained within the hydrogel, upon which the pH indicator, and thus the hydrogel matrix 18, can exhibit a change in color representative of the change in pH.

Referring again to FIG. 1, in another embodiment, the polymeric precursor can be a polymeric microsphere precursor solution 17. In this embodiment, the wound dressing can be formed to include a plurality of porous, absorbent microspheres 20 encapsulated in an absorbent substrate 22, and in one embodiment; in a crosslinked hydrogel matrix such as that described above, but that is formed without addition of a pH indicator. Of course, the absorbent substrate 22 is not limited to a hydrogel substrate, and any suitable absorbent substrate that can retain the absorbent microspheres 20 and allow visual determination of a color change of the microspheres 20 is encompassed.

Microsphere precursor solution 17 can include a biocompatible polymer and a pH indicator. In one embodiment, the biocompatible polymer of a microsphere precursor solution 17 can be a hydrogel-forming polymer as described above and the microspheres 20 can be formed of a crosslinked hydrogel polymer. For instance, the microsphere precursor solution 17 can include one or more non-toxic, biocompatible hydrogel-forming polymers that include or can be modified to include crosslinkable functionality. A suitable polymer for such an embodiment can include synthetic and/or natural hydrogel-forming polymers. By way of example, and without limitation, microsphere polymers can include alginates, collagen or derivatives thereof, cellulose or derivatives thereof, poly(lactic-co-glycolic acid) (PLGA) or derivatives thereof, polycaprolactone (PCL) or derivatives thereof, as well as combinations of different polymers, e.g., blends or copolymers thereof as discussed previously. In one embodiment, the polymer of the microspheres 17, e.g., a collagen, an alginate or another biocompatible polymer, can be selected so as to modify and control the rate of water vapor transmission from the wound surface, water absorbance, dehydration, and mechanical properties of the wound dressing that incorporates the pH indicating microspheres 17 as compared to a similar dressing that is formed without the encapsulated microspheres.

The microsphere precursor solution 17 can include a crosslinking agent and/or a crosslinking initiator as discussed previously with regard to the hydrogel precursor solution and a microsphere precursor solution 17 can include the various components in amounts as described above with regard to the hydrogel precursor solution. However, the microsphere precursor solution 17 need not include a crosslinking agent and in other embodiments, the precursor solution 17 can be crosslinked to form the microspheres upon contact with a crosslinking agent.

The microspheres 20 can be formed according to any suitable formation method including, without limitation, ultrasonic methods, mechanical methods (e.g., high energy stirring), emulsification condensation, spray condensation, etc. In one embodiment, the formation technique and/or particular characteristics of the formation technique can be designed to control the size of the microspheres. In general, however, the microspheres can be formed to any size in the micrometer scale, e.g., from about 1 micrometer to about 1 millimeter.

Modification of the size of the microspheres in conjunction with the concentration of the microspheres in the wound dressing can be utilized to control characteristics of the wound dressing including, without limitation, wicking effects including movement of fluid into the dressing. In addition, variation of microsphere size and concentration can allow for tuning of the mechanical properties (e.g., stretchability and stiffness modulus) of the wound dressing, as well as tuning of the rate of dehydration, as well as degree of swelling. Depending on the desired application and formulation characteristics, different parameters can be modified to produce the wound dressing having designed characteristics within narrow specifications.

FIG. 1 schematically illustrates one method for formation of the microspheres 20 and encapsulation of the microspheres 20 into an absorbent substrate 22. As illustrated, following formation of a microsphere precursor solution 17, an injector 24 can inject nascent spheres of the microsphere precursor solution 17 into an agitated bath 26 that includes a crosslinking agent and/or a crosslink initiator and/or under conditions to initiate crosslinking of the microsphere precursor solution 17 to form the porous absorbent microspheres 20 that incorporate a pH indicator.

The porous absorbent microspheres 20 can be distributed within an absorbent substrate 22; for instance, a hydrogel substrate 22 that can allow for visualization of a color change of the microspheres 20 encapsulated within as indicated in FIG. 1. The distribution can be carried out according to any methodology. For instance, a hydrogel precursor solution as described above save for the inclusion of a pH indicator, can be simply mixed with the crosslinked microspheres 20 prior to crosslinking of the surrounding hydrogel substrate 22. In such an embodiment, the hydrogel substrate 22 that encapsulates the microspheres 20 can be formed of the same or a different hydrogel forming polymer as that of the microspheres 20. In one embodiment, the hydrogel precursor solution can be partially solidified to form a precursor substrate, so as to prevent agglomeration of the microspheres 20 prior to final crosslinking of the precursor substrate to form the hydrogel substrate 22. By way of example, a hydrogel precursor solution can be located in a mold or cast in a desired shape (e.g., a thin film) and then dehydrated to remove at least a portion of the water of the precursor solution and solidify the precursor substrate. Following at least partial solidification, the microspheres 20 can be dispersed as desired on or in the precursor substrate.

Following combination of the porous absorbent microspheres 20 and the hydrogel precursor substrate, the hydrogel precursor substrate can be crosslinked by, e.g., addition of a crosslinking agent to the system, addition of a crosslink initiator to a system, addition of crosslinking conditions to a system, or any combination thereof. Upon crosslinking, the hydrogel substrate 22 can encapsulate a plurality of the porous absorbent microspheres 20 distributed therein.

Encapsulation of the porous, absorbent microspheres 20 into an absorbent substrate 22 can provide multiple benefits to a wound dressing, including reduction of Young's modulus and increase of elasticity. For instance, a crosslinked hydrogel substrate 22 encapsulating a plurality of pH indicating microspheres 20 can exhibit a Young's modulus of about 1 mPa or less, or about 0.5 mPa or less, in some embodiments. Incorporation of a plurality of the pH indicating microspheres 20 in an absorbent substrate 22 can also reduce dehydration rate of a wound dressing. As such, the wound dressing can remain moist at the wound site for a longer period of time as compared to moist wound dressings that exhibit fast drying rates, and as such, must be replaced more often in order to retain desired moisture.

In another embodiment, a wound dressing can incorporate the pH indicating polymeric composition in the form of fibers. For example, and as illustrated in FIG. 1, a pH indicator 10 and a biocompatible polymer 12 can be combined to form a fiber precursor 15.

Biocompatible polymers of a fiber precursor 15 can include any extrudable or solvent-dispersible polymer as generally known in the art capable of being spun, extruded, electrospun, etc. to form at least partially absorbent fibers 25 that can in turn be used in forming a fibrous substrate 26, e.g., a woven or nonwoven fibrous substrate 26 of a wound dressing. The fibers 25 can be at least somewhat absorbent so as to allow at least a portion of a fluid in contact with the fibers 25 to absorb into the body of the fibers 25 and contact the pH indicator of the polymeric composition of the fibers 25. In addition, the polymers of the fibers 25 can be suitably colored so as to allow for visual recognition of a change in color of the pH indicator of the fiber composition.

Exemplary fiber-forming polymers can include, without limitation, polyesters such as polyethylene terephthalate, polybutylene terephthalate, and polyester elastomers; polyamides such as nylon 6, nylon 6,6, nylon 6,12, poly(iminoadipoyliminohexamethylene), poly(iminoadipoyliminodecamethylene), polycaprolactam; polyurethanes such as ester-based polyurethanes and ether-based polyurethanes; fluorinated polymers such as poly(vinyl fluoride), poly(vinylidene fluoride), copolymers of vinylidene fluoride, copolymers of chlorotrifluoroethylene; chlorinated polymers; poly(butadienes); polyimides (for example, poly(pyromellitimide) and the like); polyethers; poly(ether sulfones) such as poly(diphenylether sulfone), poly(diphenylsulfone-co-diphenylene oxide sulfone); poly(sulfones); poly(vinyl esters) such as poly(vinyl acetates) and copolymers of vinyl acetate (for example, poly(ethylene-co-vinyl acetate); poly(vinyl ethers); poly(vinyl alcohols); and the like; and combinations thereof.

A fiber precursor 15 can in one embodiment include from about 0.1% w/v to about 2% w/v of the pH indicator and can include from about 50 wt. % to about 99.9 wt. % of a biocompatible polymer. However, the preferred amount of the components can vary as is known, depending upon the particular components and the formation techniques to be used. A fibrous substrate 26 that incorporates the fibers 25 can be prepared by any suitable process such as, for example, air laying techniques, spunlaid techniques such as meltblown or spunbonding, electrospinning, carding, and combinations thereof.

Upon contact of the fibrous substrate 26 with a biological fluid, such as a wound exudate, and a fiber precursor, at least a portion of the fluid can be absorbed by the fibers 25 of the substrate 26 and contact the pH indicator of the fiber composition. The color of the fibrous substrate 26 can thus change, as indicated in FIG. 1 and the change in color can be utilized to determine the pH of the biological fluid and the state of the wound.

A polymeric precursor of any type can include additives as are known in the art in standard amounts. In one embodiment, a polymeric precursor can incorporate a plasticizer, which can improve the mechanical properties and flexibility of the wound dressings. Plasticizers can also help to reduce the dehydration of wound dressings dressing. Examples of suitable plasticizers can include, without limitation, dioctylphthalate, castor oil, diacetylated monoglycerides, diethyl phthalate, glycerin, mono- and di-acetylated monoglycerides, polyethylene glycol, propylene glycol, triacetin, triethyl citrate, bis-(2-butoxyethyl) adipate, and bis-(2-ethylhexyl) sebacate polyvinyl alcohol, polyvinyl alcohol, glycerol, and polyethylene glycol. When included, a polymeric precursor can generally include a plasticizer component in an amount of from 0 to about 20 w/v % of the precursor solution.

In one embodiment, a wound dressing can be used as a vehicle for the sustained release of therapeutic agents, which encompass any agent that can enhance healing, by inclusion of the desired therapeutic agent(s) in the polymeric precursor. Therapeutic agents can include, without limitation, antimicrobial agents, antiseptic agents, anti-inflammatory agents, pain relieving agents, wound closing adhesive agents, etc. Antimicrobial agents may include, for example, sources of oxygen and/or iodine (e.g., hydrogen peroxide or a source thereof and/or an iodide salt, such as potassium iodide); antimicrobial metals, metal ions and salts, such as, for example, silver-containing antimicrobial agents (e.g., colloidal silver, silver oxide, silver nitrate, silver thiosulphate, silver sulphadiazine, or any combination thereof); or any combination thereof.

Other examples of additives of polymeric precursors can include, without limitation, surfactants, electrolytes, pH regulators, colorants, chloride sources, and mixtures thereof. Additives to the polymeric precursor can encompass materials that are retained in the wound dressing, as well as materials that are not retained in the final product. For instance, an additive, e.g., a surfactant, may serve a purpose during formation of the wound dressing, and may be removed from the other components following its intended use and during a later stage of formation of the wound dressing.

A polymeric composition that incorporates the pH indicator and a biocompatible polymer can be used as formed as a wound dressing or can be combined with other materials in formation of a wound dressing. For instance, a hydrogel or fibrous substrate that includes the polymeric composition can be combined with a backing sheet and/or an absorbent layer, e.g., a foam, a sponge, a gauze, or the like, in formation of a wound dressing. In one embodiment, the wound dressing can include the polymeric composite at a wound contacting surface of a wound dressing and an adjacent absorbent layer that can facilitate absorption of a biological fluid of interest into the polymeric composite so as to encourage contact between the biological fluid and the pH indicator. An absorptive supporting substrate can be a fibrous substrate, e.g., a woven or non-woven absorptive textile, or an absorptive non-fibrous substrate, e.g., an open-pored absorptive sponge or foam. For instance, an absorptive supporting substrate can include a fibrous wadding, e.g., a cotton wool, or other cotton-based fibers, polyester fibers, polyurethane fibers, etc.; a medical gauze, or the like or an absorbent foam, such as an open-celled hydrophilic polyurethane foam, a surgical sponge, or the like.

Wound dressings as described can be utilized in treating any wound, and in one particular embodiment, can be used in treating chronic wounds, but are not limited to such uses, and the wound dressings can be effective against many other forms of wounds and skin disease. For example, disclosed wound dressings can be effective for use as general surgical wound dressing, burn dressing, donor site dressing, bedsore dressing, and ulcer dressing, cosmetic treatment dressings, dermatology-related treatment dressings, etc. The wound dressings are suitable for partial- and full-thickness wounds with moderate to heavy exudate, and as they can exhibit an ability to retain moisture, may not require dressing changes as frequently as other, previously known wound dressings.

Exemplary wound problems that can utilize the wound dressings can include, without limitation, ulcers, sunburns, traumatic injuries, hemorrhoids, bedsores, diabetic wounds, and ischemic syndromes, such as coronary or peripheral arterial disease and angiogenesis-dependent disease. Moreover, the wound dressings can be used as the primary wound treatment protocol or in combination with other products, such as topical antibiotic treatments, debridement, etc.

In one embodiment, the wound dressings can be utilized in development of a treatment plan for a wound. For instance, a wound dressing can be located on the wound and checked periodically for color. The treatment plan can include removing or replacing the wound dressing when the color or the wound dressing indicates a basic pH. A treatment plan can include addition or modification of use of antimicrobial agents, either topical or systemic antibiotic agents, depending upon the color of the wound dressing. Moreover, in some embodiments, the color of the wound dressing can indicate the type of pathogen infecting a wound, and a treatment plan can encompass selection of particular treatment agents or protocols based upon the type of pathogen. In other embodiments, a treatment plan can include addition of agents/activities for treatment of a chronic wound.

The present disclosure may be better understood with reference to the Examples set forth below.

### Example

### Preparation of pH Indicator Solutions

Anthocyanin solution was prepared by adding 55 g of chopped red cabbage to 90 mL of DI water, keeping at 90° C. for 1 hour, and filtering to obtain 90 mL of the final solution. About 0.1 g litmus dye (TCI, Tokyo, Japan) was added in 15 mL DI water and stirred to complete dilution. 0.8% w/v and 1.5% w/v bromothymol blue solutions were prepared by dissolving the dye in water.

Preparation of pH-Sensitive Alginate Microspheres

A solution of sodium alginate/glycerol (2% w/v-10% w/v) (Sigma-Aldrich-Fisher Scientific) was prepared in anthocyanin solution and homogenized at 800 rpm using stirrer for 1 hour. Homogenized solution was put into a sonication bath to remove the trapped air bubbles. Additionally, alginate/glycerol (2% w/v-10% w/v) containing 0.5% w/v litmus solution was prepared and homogenized for 1 hour. Both solutions were loaded into a syringe separately and ejected from a 25-gauge needle into agitated bath of 200 mL of 2% (w/v) CaCl2). (Sigma-Aldrich) as cross linker. The calcium-cross-linked alginate beads kept in the curing bath for 20 minutes, subsequently filtered and rinsed with DI water to remove excess CaCl2).

Fabrication of pH-Indicating Hydrogel Substrate

Sodium alginate hydrogel substrate was prepared by casting-solvent evaporation technique. A solution of alginate/glycerol (2% w/v-10% w/v) was prepared and casted on a Petri dish or a mold and oven-dried at 40° C. for 4 hours to make a solid gel. Microspheres impregnated with pH indicator were added as a second layer to the alginate gel followed by adding more alginate/glycerol solution and oven dried overnight. To crosslink and form an alginate substrate, an aqueous solution of CaCl₂) 2% w/v was poured into the mold or petri dish and left at room temperature for 20 minutes to solidify. Crosslinked sheets were removed and immersed in DI water for 5 minutes to remove excess CaCl.

Fabrication of pH-Indicating Fibrous Substrate

Thermoplastic polyurethane nanofiber matrix, with a moderately absorptive capacity was designed with spinning method. Polymer solution of thermoplastic polyurethane/bromothymol blue was prepared in dimethyl formamide (DMF) and transferred to an electrospinning syringe with the needle connected to the positive terminal of a high voltage supply. The electrospinning was performed by applying high voltage and a nanofiber mat was collected on aluminum foil. The resulting polymer fiber was flexible and elastic, with a high tensile strength.

Analysis of pH-Indicating Substrates in Response to Basic pH Environments

A glass microelectrode pH meter (VWR, USA) was used to measure the pH of solutions of 0.1 M potassium phosphate buffer with different pH (6.6, 7.1, 7.4, 8.0, and 9). 200 μL of buffer was added to each substrate type and color change was recorded.

### Colorimetric Detection of Bacterial Infections

Strains of Gram-negative Pseudomonas aeruginosa and E. coli and Gram-positive Staphylococcus aureus were cultured in Tryptic soy broth. Strains selected are highly prevalent in wounds and infections caused by these pathogens remain a common complication in acute and chronic wounds. Following a 24-hour bacterial incubation, 200 μL bacteria in culture was deposited on the pH sensors patch to do photography.

### Ex Vivo Assessment of the pH Indicator Mat Using the Porcine Dermal Biofilm Model

The pH indicator nanofiber mat was assessed in a similar environment to human skin by using an ex vivo porcine dermal model. This model is based on pig skin that shares similar anatomy and physiology to human skin. The model can simulate and resemble wound infection and biofilm formation in human wounds and the result can be correlated to human. Porcine dermis was procured from a USDA facility with a specific thickness of approximately 2.5 mm. The dermal tissue was then cut into explants and an artificial “wound” was created by removing the epidermis using a scalpel. The tissue was then extensively washed and sterilized using chlorine gas. The explants were then inoculated with approximately 10⁵⁻⁶ CFU of bacteria (E. coli and P. aeruginosa) per explant and allowed to incubate for 3 days on 0.5% TSA containing antibiotic with daily transfer to fresh agar plates to prevent overgrowth of bacteria on agar. Following a 72-hour biofilm growth period, the explants were washed three times with saline prior to assessment of the polymer films. After the wash, the polymer films were applied to the tissue surface to ensure contact between the ex vivo wound and the polymer film for up to 1 minute. The bacteria present on the surface of the tissue were recovered using a scrubbing technique in recovery media, followed by serial dilution and plating on standard 1.5% TSA plates. Colonies were counted after 18-24 hours of incubation.

### Cytotoxicity Assay (MTS)

In order to determine cell viability, normal human dermal fibroblasts (NHDFs) were cultured in their growth media DMEM/10% FBS to reach 80% confluency. Cells were seeded at the density of 1×10⁵ cells per 6-well plate in a total volume of 2 mL in each well. Cells were incubated at 37° C. and 5% CO₂ to allow for cell attachment and reach confluency. pH indicator substrates were sterilized through UV radiation for 15 minutes and were placed on cells, growing in culture for 24 hours. The reaction and toxicity were observed with a microscope and the fixed samples were stained with DAPI-Actin to visualize cell nucleus and cell cytoskeleton.

A direct method of determining toxicity was carried out by extracting the test materials with cell culture media in a 37° C. incubator for 24 hours. Eluates from the test materials were added to the cells and exposed for 24 hours; untreated cells were used as control. Following 24 hours of incubation, media containing 20% MTT solution was replaced with eluate media and incubated for 2 hours. Absorbance was read at 490 nm using a Spectramax® 190 spectrophotometer and cell viability was assessed.

Toxicity assay of the human dermal fibroblast (HDF) treated with different concentrations of pH indicator was separately investigated.

A release study of leachable material of prototype substrates was done in compliance with biocompatibility regulation in saline and DMEM cell culture media for 24 hours at 37° C.

### Experimental Results

The results of skin simulated pH testing were demonstrated using phosphate buffer and bacteria in culture with pH 8-9.

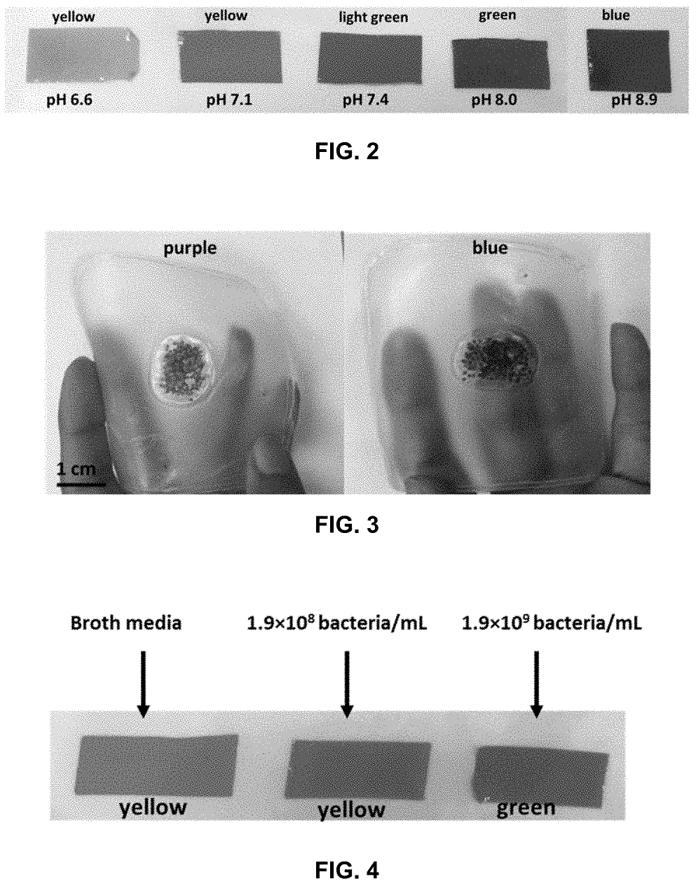

FIG. 2 illustrates the gradient of color change from yellow to blue observed for the bromothymol blue-containing fibrous substrate when exposed to phosphate buffer with different pH (6.6, 7.1, 7.4, 8.0, and 9). The substrate was originally yellow and at acidic environment did not change color, but increasing pH demonstrated color change to light green, dark green and blue.

FIG. 3 illustrates the color of a litmus pH-indicating substrate as formed (left hand image) with a purple color and the color change to blue in response to phosphate buffer pH 9.2 (right hand image).

FIG. 4 illustrates a bromothymol blue-containing fibrous substrate following deposition of broth media alone (left), E. coli bacteria supernatant at concentration of 1.9×10⁸ cell/mL (center), and E. coli bacteria supernatant at concentration of 1.9×10⁹ cell/mL (right) on the surface of the substrate. The substrates contacted with the bacteria supernatant demonstrated color change from a light yellow in 10 seconds. The substrate contacted with broth media only did not show any change in color. Increasing the bacteria concentration made a significant color change from dark yellow to green.

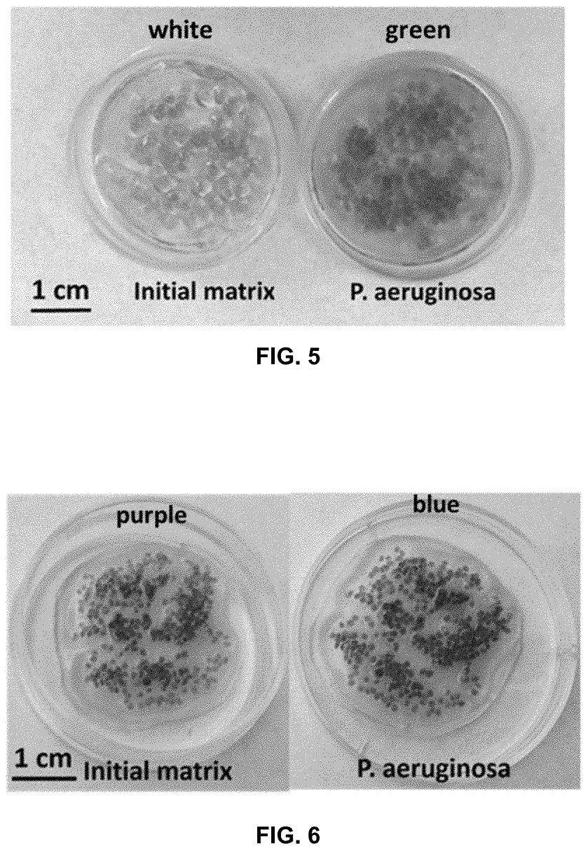

FIG. 5 illustrates an anthocyanin pH-indicating hydrogel substrate as formed (left hand image) and the color change of the substrate from white to green in response to P. aeruginosa (right hand image). 200 μL of bacteria in culture was added to the substrate and the color change was photographed after 1 hour. The Anthocyanin beads in the substrate turned green when exposed to bacteria at basic pH 8-9.

FIG. 6 illustrates the color of a litmus containing pH-indicating substrate as formed (left hand image) and the color change from purple to blue in response to P. aeruginosa (right hand image). 200 μL of bacteria in culture was added to the substrate and the color change was photographed after 1 hour. The litmus-containing beads were red at pH<7, and when they were exposed to bacteria at basic pH 8-9, they turned blue.

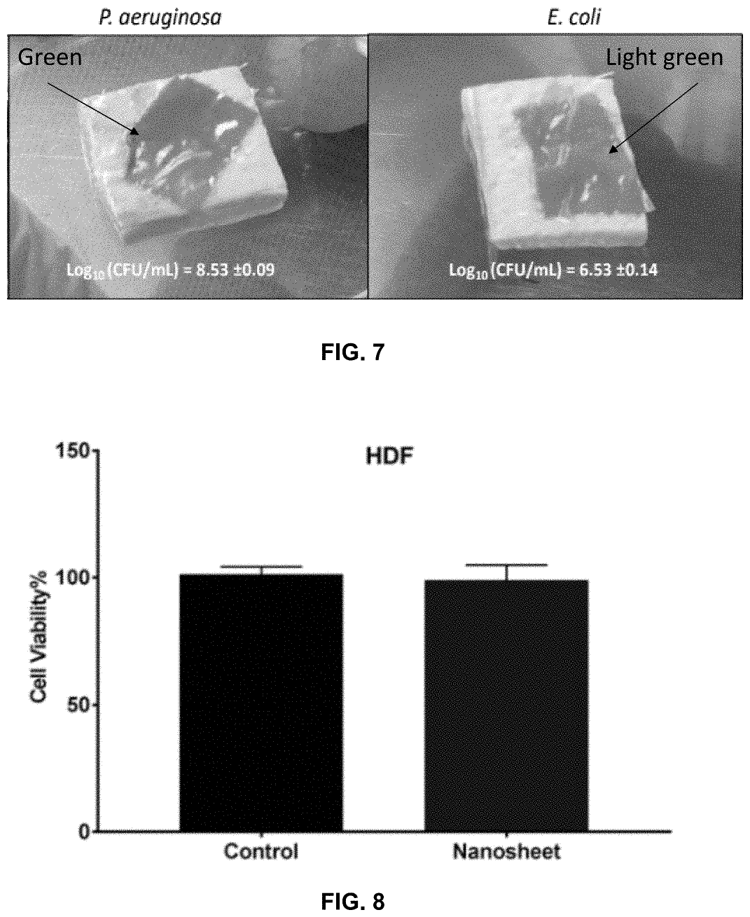

FIG. 7 illustrates the response of a nanofiber polymer mat to biofilm-associated bacteria within the ex vivo model within 1 minute. The pH indicator mat turned green in response to P. aeruginosa and changed to light green in response to E. coli biofilm as compared to the original yellow mat. Cell counts determined after the visual assessments were Log 10 (CFU/mL)=8.53±0.09 for P. aeruginosa and Log₁₀ (CFU/mL)=6.53±0.14 for E. coli.

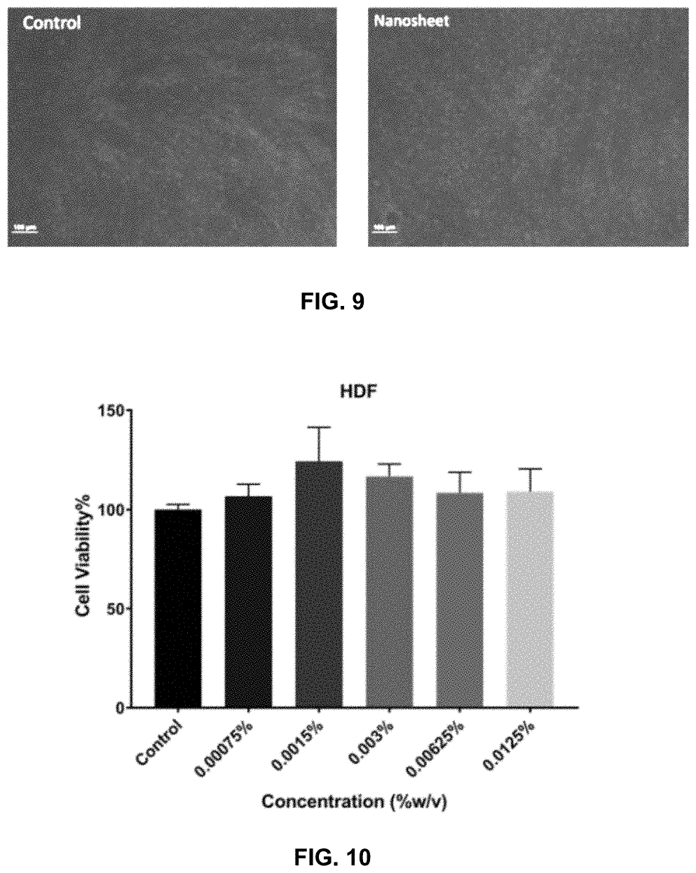

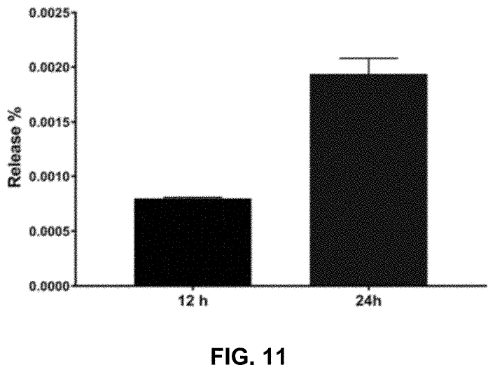

FIG. 8-FIG. 11 illustrate the results of toxicity assays of a fibrous substrate carried out according to two different methods. As indicated in FIG. 8, HDF cell treatment with eluates for 24 hours did not show toxicity as compared to control (non-treated cells) in MTT assay. FIG. 9 presents microscopic images of the control and tested substrate. As illustrated, the results demonstrated no toxicity for the cells that were exposed to the pH-indicating substrate as compared to the control. FIG. 10 presents the results of a toxicity assay of human dermal fibroblasts treated with different concentrations of a pH indicator. As demonstrated exposure to a safe concentration of the pH indicator (based on FDA regulation) for 24 hours (≤0.02% w/v indicator in polymer solution) did not indicate cell toxicity. Upon extraction of the substrate with saline media, there was essentially no release of pH indicator. However, as indicated in FIG. 11, there was limited release quantified at a concentration of 0.002% following extraction with DMEM cell culture media for 24 hours.

While certain embodiments of the disclosed subject matter have been described using specific terms, such description is for illustrative purposes only, and it is to be understood that changes and variations may be made without departing from the spirit or scope of the subject matter.

What is claimed is:

1. A wound dressing comprising:
a wound contacting surface; and
an absorbent substrate at the wound contacting surface, the absorbent substrate comprising a polymeric composition, the polymeric composition comprising a biocompatible polymer and a pH indicator, the pH indicator exhibiting a detectable color change in the visible spectrum that is indicative of the pH of a fluid in contact with the polymeric composition.

2. The wound dressing of claim 1, the absorbent substrate comprising fibers, the fibers comprising the polymeric composition.

3. The wound dressing of claim 1, the absorbent substrate comprising a crosslinked hydrogel matrix, the crosslinked hydrogel matrix comprising the polymeric composition.

4. The wound dressing of claim 1, the absorbent substrate encapsulating a plurality of microspheres, the microspheres comprising the polymeric composition.

5. The wound dressing of claim 1, the polymeric composition further comprising a plasticizer.

6. The wound dressing of claim 5, the plasticizer comprising dioctylphthalate, castor oil, diacetylated monoglycerides, diethyl phthalate, glycerin, mono- and di-acetylated monoglycerides, polyethylene glycol, propylene glycol, triacetin, triethyl citrate, bis-(2-butoxyethyl) adipate, and bis-(2-ethylhexyl) sebacate polyvinyl alcohol, polyvinyl alcohol, glycerol, or polyethylene glycol.

7. The wound dressing of claim 1, the biocompatible polymer comprising an alginate, a collagen, a gelatin, a cellulose, a poly(lactic-co-glycolic acid), a polycaprolactone, a polyester, a polyamide, a polyurethane, a fluorinated polymer, a poly(butadiene), a polyimide, a polyether, a poly(vinyl ester), a poly(vinyl ethers), a poly(vinyl alcohol), or derivatives, copolymers, or combinations thereof.

8. The wound dressing of claim 1, the pH indicator comprising litmus, anthocyanin, nitrazine yellow, brilliant yellow, cresol red, bromocresol purple, chlorophenol red, bromothymol blue, thymol blue, bromoxylenol blue, neutral red, phenol red, xylenol blue, m-cresol purple, orcein, erythrolitmin (or erythrolein), azolitmin, spaniolitmin, leucoorcein, leucazolitmin, or any combination thereof.

9. The wound dressing of claim 1, the polymeric composition further comprising a therapeutic agent.

10. A method for forming a wound dressing comprising:
forming a polymeric precursor, the polymeric precursor comprising a pH indicator and a biocompatible polymer;
solidifying the polymeric precursor to form a polymeric composition that incorporates the pH indicator and the biocompatible polymer;
forming an absorbent substrate, the absorbent substrate comprising the polymeric composition, wherein the absorbent substrate is at a wound contacting surface of the wound dressing.

11. The method of claim 10, wherein the step of solidifying the polymeric precursor comprises crosslinking the polymeric precursor.

12. The method of claim 10, wherein the step of solidifying the polymeric precursor comprises forming a plurality of microspheres comprising the polymeric composition.

13. The method of claim 12, the method further comprising distributing the plurality of microspheres in the absorbent substrate.

14. The method of claim 10, wherein the step of solidifying the polymeric precursor comprises forming a plurality of fibers, the fibers comprising the polymeric composition.

15. The method of claim 10, further comprising locating the absorbent substrate adjacent a supporting substrate of the wound dressing.

16. A method for treating a wound comprising:
applying a wound contacting surface of a wound dressing to the wound, the wound contacting surface comprising an absorbent substrate, the absorbent substrate comprising a polymeric composition, the polymeric composition comprising a biocompatible polymer and a pH indicator; and
following a period of time, visually examining the wound contacting surface to determine a color of the pH indicator, the color indicating the pH of a biological fluid of the wound.

17. The method of claim 16, wherein the wound is a chronic wound, a burn wound, or an acute wound.

18. The method of claim 16, the pH of the biological fluid indicating a bacterial infection in the wound, the method further comprising initiating a wound treatment comprising use of an antimicrobial agent.

19. The method of claim 16, further comprising initiating a wound treatment protocol based upon the status of the wound as determined from the indicated pH of the biological fluid.

20. The method of claim 16, further comprising repeating the process to determine a dynamic state of the wound.
